(19) United States
(12) Patent Application Publication
(10) Pub. No.: US 2017/0233742 A1
(43) Pub. Date: Aug. 17, 2017

(54) Compositions Comprising Small Interfering RNA Molecules for Prevention and Treatment of Ebola Virus Disease

(71) Applicant: Sirnaomics, Inc., Gaithersburg, MD (US)

(72) Inventors: Yibin Cai, Gaithersburg, MD (US); Patrick Y. Lu, Potomac, MD (US); John J. Xu, Germantown, MD (US)

(73) Assignee: Sirnaomics, Inc., Gaithersburg, MD (US)

(21) Appl. No.: 15/518,706

(22) PCT Filed: Oct. 16, 2015
(86) PCT No.: PCT/US2015/056085

§ 371 Date: Apr. 12, 2017

Related U.S. Application Data
(60) Provisional application No. 62/065,523, filed on Oct. 17, 2014.

(51) Int. Cl. C12N 15/113; C12Q 1/70; A61K 49/00

(52) U.S. Cl. CPC C12N 15/1131; A61K 49/0008; C12Q 1/701; C12N 2310/14; C12N 2320/31; C12Q 2600/178; C12Q 2600/136

(57) ABSTRACT

Disclosed herein are small interfering RNA (siRNA) molecules and pharmaceutical compositions containing them for the prevention and treatment of Ebola virus disease. The present invention provides siRNA molecules that inhibit Ebola virus gene expression, compositions containing the molecules, and methods of using the molecules and compositions to prevent or treat EVD in a subject, such as a human patient.

### CROSS-REFERENCE TO RELATED PATENT APPLICATIONS

This application claims the benefit of and priority to U.S. Provisional Patent Application No. 62/065,523, filed Oct. 17, 2014.

### FIELD OF INVENTION

The invention relates to compositions comprising small interfering RNA (siRNA) molecules for the prevention and treatment of Ebola virus disease.

### BACKGROUND

### 1. EBOLA Virus Disease: Biology and Pathology

Ebola virus disease (EVD) is a severe, often fatal illness in humans and primates, leading to a fatality rate as high as 50% to 90% of those infected with the virus. A total of 5 subtypes of Ebola virus has been identified since it first appeared in 1976 in Congo and Sudan: Zaire ebolavirus (EBOV or ZEBOV), Sudan ebolavirus (SUDV), Tai Forest ebolavirus (TAFV), Reston ebolavirus (RESTV) and Bundibugyo ebolavirus (BDBV). Amongst them, EBOV, SUDV and BDBV have been identified to be responsible for the large EVD outbreaks in Africa.

EVD patients usually have an incubation period of 2 to 21 days. Stage I of the disease is not very specific and has been observed as extreme asthenia, diarrhea, nausea and vomiting, with anorexia, abdominal pain, headaches, arthralgia (neuralgic pain in joints), myalgia, back pain, mucosal redness of the oral cavity, and dysphagia. At stage II, the symptoms are more distinguished with hemorrhaging, neuropsychiatric abnormalities, anuria, and hiccups. The current knowledge regarding the transmission between human and human is not very clear but has been characterized through close contact with the bodily fluids of infected individuals.

Ebola is a single-stranded, linear, negative-sense RNA virus, belonging to Filoviridae family (filovirus). Genus Ebolavirus comprises 5 distinct species: EBOV, SUDV, TAFV, RESTV, and BDBV.

The Ebola genome is approximately 19 kb in length. It encodes seven structural proteins—nucleoprotein (NP), polymerase cofactor VP35, VP40, Glycoprotein (GP), transcription activator VP30, VP24, and RNA polymerase (L)—and one non-structural protein secreted Glycoprotein (sGP).

Ebola virus seems to be most active in infecting fibroblasts of any type (especially fibroblastic reticular cells). The next most frequent cell types are mononuclear phagocytes with dendritic cells more affected than monocytes or macrophages. Endothelial cells become infected after the connective tissue surrounding them is fully involved. Then, almost as a final insult, epithelial cells of any type are infected. In general, epithelial cells become infected only if they contact other cells that amplify the virus such as fibroblastic reticular cells (FRC) and mononuclear cells. This would be true for skin appendages like hair follicles and sweat glands because they are heavily vascularized and have a lot of FRC networks associated with them. Liver cells and adrenal gland epithelial cells have fibroblastic reticulum as their main connective tissue and both have resident mononuclear cell phagocytes hanging on FRC cells near the blood/epithelial cell interface. The time sequence of infection and spread of Ebola is difficult to speculate on because most studies have been conducted on “autopsied” animals, i.e. they are at the end stage of infection.

Currently, there is no licensed vaccine or specific treatment for preventing or treating EDV. The present invention addresses this need.

### 2. Current Prophylaxis and Therapeutics

There have been many attempts to develop potential therapies and vaccines for EVD. Here are some of the leading programs listed by WHO in a recent report:

Convalescent plasma: Studies suggest blood transfusions from EVD survivors might prevent or treat Ebola virus infection in others, but the results of the studies are difficult to interpret. It is not known whether antibodies in the plasma of survivors are sufficient to treat or prevent the disease. This approach is safe if provided by well-managed blood banks Risks are like those associated with the use of any blood products, such as the transmission of blood-borne pathogens that cause disease. There is a theoretical concern about antibody dependent enhancement of EVD infection, which can increase infectivity in the cells. Blood transfusion is culturally acceptable in West Africa. Potential donors are Ebola survivors, but the logistics of blood collection are an issue. Options to conduct studies in patients are being explored. The first batches of convalescent plasma might be available by the end of 2014.

ZMapp Cocktail: This therapeutic candidate has three chimeric mouse-human monoclonal antibodies (Mapp Biopharmaceutical Inc.). The three antibodies in this mixture block or neutralize the virus, by binding to or coating a different site on the covering or “envelope” of the virus. Studies in monkeys showed a strong survival up to five days after infection, when virus and/or fever were present. There have been no formal safety studies in humans. Very small numbers of EVD-infected people have been given ZMapp on a compassionate basis, and no safety issues have been reported to date. Clinical effectiveness is still uncertain. A very limited supply (fewer than 10 treatment courses) has been deployed to the field. Efforts to scale up production may yield increased supplies of potentially few hundred doses by the end of 2014.

Hyperimmune globulin: This therapeutic product candidate is prepared by purifying and concentrating plasma of immunised animals or previously infected humans with high titres of neutralizing antibody against EVD. Antibodies that can neutralize the different EVD strains have been produced and shown to be protective in monkeys when treatment begins 48 hours after exposure to EVD. It is generally safe. There has been extensive experience with the use of hyperimmune globulin against other infectious agents in humans. Inactivation and purification procedures effectively eliminate blood-borne pathogens that cause disease. It is not currently available. Several months are needed to immunize animals, collect plasma and make the purified product. Work is starting on the production of immune globulin in horses, and of human immune globulin in cattle. Studies in horses could take place within six months, but large-scale batches for use in humans are not expected before mid-2015.

TKM-100802: This product candidate is a lipid nanoparticle small interfering ribonucleic acid based therapeutic by Tekmira. The drug targets two essential viral genes to stop the virus from replicating. It is effective in guinea pigs and monkeys. In monkeys, there is 83% survival if administered 48 hours after infection, and 67% survival 72 hours after infection. A single-dose study in healthy volunteers found side effects, including headache, dizziness, chest tightness and raised heart rate at high doses. At lower doses projected to be the dose used for treatment, drug was better tolerated. The US Food and Drug Administration has authorized emergency use in EVD-infected patients. A limited number of treatment courses are potentially available. There is potential for the production of 900 courses by early 2015.

AVI 7537: This therapeutic candidate is an oligonucleotide-based phosphorodiamidate (Sarepta). In monkey studies, doses of 14 to 40 mg/kg for 14 days showed typical survival ranging from 60% to 80% when given at the time of infection. Human tolerability has been demonstrated in early studies. The active pharmaceutical ingredient is available for 20 to 25 courses by mid-October. The potential production of approximately 100 treatment courses will be completed by early 2015.

Favipivavir/T-705: This therapeutic candidate (Toyama Chemical/Fuji Film) has shown effectiveness against EVD in mice, but in a monkey study only one out of six survived. Another study of animals using a different dose regimen is underway. Approved in Japan for Ebola treatment under special circumstances, this drug remains under study in other countries. It has been tested in more than 1000 people, with no major adverse effects reported. But the dose proposed for treatment of EVD could be 2-5 times higher than that tested so far and duration of treatment might be longer than in current Ebola studies. It should not be used during pregnancy due to potential birth defects. It has not been studied in humans for Ebola. Use for field post-exposure prophylaxis is under discussion. More than 10,000 treatment courses may available, pending determination of the dose for treatment of EVD.

BCX4430: Studies (Biocryst) of this anti-viral in animals indicate 83% to 100% survival in rodents with EVD. It is also effective in animals 48 hours after infection with the lethal Marburg virus, which belongs to the same family as Ebola. Testing for EVD in monkeys is underway and there is no human safety study or data available. Safety studies are planned. It needs animal treatment and protection data for EVD before it can be considered. No material is currently available for field use.

Interferons: This commercially available product can induce an antiviral state in exposed cells and regulates the immune system. A study showed delayed time to death in monkeys but no overall increased survival for EVD patients. Early administration enhances the effectiveness of treatment in animals and lengthens the time after the viral infection at which antibodies show effectiveness. Various forms are approved for treating chronic hepatitis and multiple sclerosis. Higher doses are associated with increased adverse effects but no greater efficacy. There are several types of commercially available interferons. Decisions regarding which one to use, when to use, and the dose regimen need careful consideration.

3. siRNA

RNA interference (RNAi) is a naturally occurring, highly specific mode of gene regulation. The mechanics of RNAi are both exquisite and highly discriminating (Siomi and Siomi 2009). At the onset, short (19-25 bp) double stranded RNA sequences (referred to as short interfering RNAs, siRNAs) associate with the cytoplasmically localized RNA Interference Silencing Complex (RISC) (Jinek et al. 2009). The resultant complex then searches messenger RNAs (mRNAs) for complementary sequences, eventually degrading (and/or attenuating translation of) these transcripts. Scientists have co-opted the endogenous RNAi machinery to advance a wide range of basic studies.

4. Challenges Associated with siRNA Therapeutic Development—Delivery Barriers.

There are many biological barriers and factors that protect the lungs from foreign particles, such as a thick and elastic mucus layer that may bind inhaled drugs and remove them via mucus clearance mechanism, low basal and stimulated rates of endocytosis on the apical surfaces of well-differentiated airway epithelial cells, the presence of RNase extra- and intra-cellularly, and the presence of endosomal degradation systems in the target cells, among others. Overcoming the difficulties concerning respiratory tract delivery and effective cellular entry and function will pave the way for siRNA as a systemic EVD therapeutic.

In addition, the delivery vehicle and mode of administration must be chosen to be appropriate to the stage of the infection and provide the fastest onset of silencing at the required site of action, e.g. early stage infection/prophylaxis at the fibroblasts and endothelial cells, later stage disease through systemic administration. Among them, systemic delivery through IV infusion has been shown to be the most effective way to treat the disease. In a pandemic setting, this delivery approach will allow ease of administration directly by patients.

### BRIEF DESCRIPTION OF THE FIGURES

FIG. 1. Genome Structure of Ebola Virus (Zaire Subtype). The large arrows indicate the targeted genes for siRNA design: VP24, VP30, VP35, VP40 and LP genes. The smaller arrows indicate individual siRNAs against each targeted viral gene.

FIG. 2. Histidine-Lysine Polymer (HKP) Carrier for siRNA Delivery. HKP is a validated polymeric carrier for efficient siRNA delivery in vivo, including both local and systemic administrations. This Polymer Nanoparticle (PNP) is relatively easily formulated with an siRNA payload.

FIG. 3. SLiC/siRNA Lipid Nanoparticle Formulations. We have developed a novel lipid nanoparticle (LNP) with a spermine head and two lipid tails conjugated with cholesterol. This carrier has been tested for in vitro transfection of siRNA oligos into cells and in vivo delivery to mouse models.

FIG. 4. Tissue Specific siRNA Delivery System Targeting Endothelial Cells. We have developed histidine-lysine (HK):siRNA nanoplexes modified with PEG and a cyclic RGD (cRGD) ligand targeting αvβ3 and αvβ5 integrins.

### SUMMARY OF THE INVENTION

The present invention provides siRNA molecules that inhibit Ebola virus gene expression, compositions containing the molecules, and methods of using the molecules and compositions to prevent or treat EVD in a subject, such as a human patient.

### SiRNA Molecules

As used herein, an “siRNA molecule” or an “siRNA duplex” is a duplex oligonucleotide, that is a short, double-stranded polynucleotide, that interferes with the expression of a gene in a cell, after the molecule is introduced into the cell, or interferes with the expression of a viral gene. For example, it targets and binds to a complementary nucleotide sequence in a single stranded (ss) target RNA molecule. SiRNA molecules are chemically synthesized or otherwise constructed by techniques known to those skilled in the art. Such techniques are described in U.S. Pat. Nos. 5,898,031, 6,107,094, 6,506,559, 7,056,704 and in European Pat. Nos. 1214945 and 1230375, which are incorporated herein by reference in their entireties.

The siRNA molecule of the invention is an isolated siRNA molecule that targets and binds to a conserved region of an Ebola virus gene. As used herein, “target” or “targets” means that the molecule binds to a complementary nucleotide sequence in an Ebola virus gene, which is genomic RNA molecule, or it binds to mRNA produced by the gene or to viral complementary RNA. This inhibits or silences the expression of the viral gene and/or its replication. As used herein, a “conserved region” of an Ebola virus gene is a nucleotide sequence that is found in more than one strain of the virus, is identical among the strains, rarely mutates, and is important for viral infection and/or replication and/or release from the infected cell.

In one embodiment, the siRNA molecule is a double-stranded oligonucleotide with a length of 17 to 35 base pairs. In one aspect of this embodiment, the molecule is a double-stranded oligonucleotide with a length of 19 to 27 base pairs. In another aspect of this embodiment, it is a double-stranded oligonucleotide with a length of 21 to 25 base pairs. In still another aspect of this embodiment, it is a double-stranded oligonucleotide with a length of 21 base pairs. In a further aspect of this embodiment, it is a double-stranded oligonucleotide with a length of 25 base pairs. In all of these aspects, the molecule may have blunt ends at both ends, or sticky ends with overhangs at both ends (unpaired bases extending beyond the main strand), or a blunt end at one end and a sticky end at the other. In one particular aspect, it has blunt ends at both ends. In one particular aspect, the molecule has a length of 21 base pairs (21 mer) with a dtdt overhang. In another particular aspect, the molecule has a length of 25 base pairs (25 mer) and has blunt ends at both ends.

One or more of the ribonucleotides comprising the molecule can be chemically modified by techniques known in the art. In addition to being modified at the level of one or more of its individual nucleotides, the backbone of the oligonucleotide can be modified. Additional modifications include the use of small molecules (e.g. sugar molecules), amino acids, peptides, cholesterol, and other large molecules for conjugation onto the siRNA molecule.

The siRNA molecules target the genes of any Ebola virus. Such viruses include Zaire ebolavirus (EBOV), Sudan ebolavirus (SUDV), Tai Forest ebolavirus (TAFV), Reston ebolavirus (RESTV), and Bundibugyo ebolavirus (BDBV).

In one embodiment, the conserved Ebola virus regions are the genes (RNA sequences) for VP24, VP30, VP35, VP40, and L Polymerase or any part thereof. Examples of such genes are disclosed in Table 1.

Particular siRNA sequences that represent some of the siRNA molecules of the invention are disclosed in Tables 2-6.

The siRNA molecules of the invention also include ones derived from those listed in Tables 2-6 and otherwise herein. The derived molecules can have less than the 21 or 25 base pairs shown for each molecule, down to 17 base pairs, so long as the “core” contiguous base pairs remain. That is, once given the specific sequences shown herein, a person skilled in the art can synthesize molecules that, in effect, “remove” one or more base pairs from either or both ends in any order, leaving the remaining contiguous base pairs, creating shorter molecules that are 24, 23, 22, 21, 20, 19, 18, or 17 base pairs in length, if starting with the 25 base pair molecule or 20, 19, 18, or 17 base pairs in length, if starting with the 21 base pair molecule. For example, the derived molecules of the 25 mer molecules disclosed in Tables 2-6 include: a) 24 contiguous base pairs of any one or more of the molecules; b) 23 contiguous base pairs of any one or more of the molecules; c) 22 contiguous base pairs of any one or more of the molecules; b) 21 contiguous base pairs of any one or more of the molecules; d) 20 contiguous base pairs of any one or more of the molecules; e) 19 contiguous base pairs of any one or more of the molecules; f) 18 contiguous base pairs of any one or more of the molecules; and g) 17 contiguous base pairs of any one or more of the molecules. It is not expected that molecules shorter than 17 base pairs would have sufficient activity or sufficiently low off-target effects to be pharmaceutically useful; however, if any such constructs did, they would be equivalents within the scope of this invention.

Alternatively, the derived molecules can have more than the 21 or 25 base pairs shown for each molecule, so long as the 21 or 25 contiguous base pairs remain. That is, once given the specific sequences disclosed herein, a person skilled in the art can synthesize molecules that, in effect, “add” one or more base pairs to either or both ends in any order, creating molecules that are 26 or more base pairs in length and containing the original 25 contiguous base pairs or creating molecules that are 22 or more base pairs in length and containing the original 21 contiguous base pairs.

The siRNA molecule may further comprise an immune stimulatory motif. Such motifs can include specific RNA sequences such as 5′-UGUGU-3′ (Judge et al., Nature Biotechnology 23, 457-462 (1 Apr. 2005)), 5′-GUCCUUCAA-3′ (Hornung et al., Nat. Med. 11,263-270(2005). See Kim et al., Mol Cell 24; 247-254 (2007). These articles are incorporated herein by reference in their entireties. These are siRNA sequences that specifically activate immune responses through Toll-like receptor (TLR) activation or through activation of key genes such as RIG-I or PKR. In one embodiment, the motif induces a TH1 pathway immune response. In another embodiment, the motif comprises 5′-UGUGU-3′, 5′-GUCCUUCAA-3′, 5′-GGGxGG-3′ (where x is A, T, G and C), or CpG motifs 5′-GTCGTT-3′.

### Pharmaceutically Acceptable Carriers

Pharmaceutically acceptable carriers include saline, sugars, polypeptides, polymers, lipids, creams, gels, micelle materials, and metal nanoparticles. In one embodiment, the carrier comprises at least one of the following: a glucose solution, a polycationic binding agent, a cationic lipid, a cationic micelle, a cationic polypeptide, a hydrophilic polymer grafted polymer, a non-natural cationic polymer, a cationic polyacetal, a hydrophilic polymer grafted polyacetal, a ligand functionalized cationic polymer, a ligand functionalized-hydrophilic polymer grafted polymer, and a ligand functionalized liposome. In another embodiment, the polymers comprise a biodegradable histidine-lysine polymer, a biodegradable polyester, such as poly(lactic acid) (PLA), poly(glycolic acid) (PGA), and poly(lactic-co-glycolic acid) (PLGA), a polyamidoamine (PAMAM) dendrimer, a cationic lipid, or a PEGylated PEI. Cationic lipids include DOTAP, DOPE, DC-Chol/DOPE, DOTMA, and DOTMA/DOPE. In still another embodiment, the carrier is a histidine-lysine copolymer that forms a nanoparticle with the siRNA molecule, wherein the diameter of the nanoparticle is about 100 nm to about 400 nm. In a further embodiment, the ligand comprises one or more of an RGD peptide, such as H-ACRGDMFGCA-OH, an RVG peptide, such as H-YTIWMPENPRPGTPCDIFTNSRGKRASNG-OH, or a FROP peptide, such as H-EDYELMDLLAYL-OH.

In one embodiment, the carrier is a polymer. In one aspect of this embodiment, the polymer comprises a histidine-lysine copolymer. Such copolymers are described in U.S. Pat. Nos. 7,070,807 B2 and 7,163,695 B2, which are incorporated herein by reference in their entireties.

In another embodiment, the carrier is a liposome. In one aspect of this embodiment, the liposome comprises a cationic lipid conjugated with cholesterol. In a further aspect, the cationic lipid comprises a spermine head and one or two oleyl alcoholic tails. In a further aspect, the liposome comprises Spermine-Liposome-Cholesterol.

### Pharmaceutical Compositions

The invention includes a pharmaceutical composition comprising an siRNA molecule that targets a conserved region of an Ebola virus gene and a pharmaceutically acceptable carrier. In one embodiment, the carrier condenses the molecules to form a nanoparticle. The compositions may be lyophilized into a dry powder.

In one embodiment, the composition comprises at least two different siRNA molecules that target one or more conserved regions of an Ebola virus gene or one or more conserved regions of different Ebola virus genes and a pharmaceutically acceptable carrier. The genes can be in the same virus strain or in two different virus strains. The composition can include one or more additional siRNA molecules that target still other Ebola virus genes in the same virus strain or in different virus strains. In one aspect of this embodiment, the composition is a nanoparticle.

In another embodiment, the composition comprises at least three different siRNA molecules that target one or more conserved regions of an Ebola virus gene or one or more conserved regions of different Ebola virus genes and a pharmaceutically acceptable carrier. The genes can be in the same virus strain or in different virus strains. The composition can include one or more additional siRNA molecules that target still other Ebola virus genes in the same virus strain or in different virus strains. In one aspect of this embodiment, the composition is a nanoparticle.

In one particular embodiment, the pharmaceutical composition comprises at least two different siRNA molecules that target one or more conserved regions of an Ebola virus gene (RNA) or one or more conserved regions of different Ebola virus genes (RNAs) and a pharmaceutically acceptable carrier, wherein the Ebola virus is selected from the group consisting of Zaire ebolavirus (EBOV), Sudan ebolavirus (SUDV), Tai Forest ebolavirus (TAFV), Reston ebolavirus (RESTV), and Bundibugyo ebolavirus (BDBV), wherein the conserved regions of Ebola virus genes (RNAs) are selected from the group consisting of VP24, VP30, VP35, VP40, and L Polymerase genes (RNAs) or parts thereof, wherein the pharmaceutically acceptable carrier comprises either a polymer or a liposome, and wherein the siRNA molecules and carrier form a nanoparticle. In one aspect of this embodiment, the siRNA molecules comprise 25 mer blunt-end siRNA molecules and the carrier is Histidine-Lysine copolymer or Spermine-Liposome-Cholesterol. In another aspect of this embodiment, the siRNA molecules comprise 21 mer siRNA molecules with a dtdt overhang and the carrier is Histidine-Lysine copolymer or Spermine-Liposome-Cholesterol. In a further aspect of this embodiment, the gene targets are VP30, VP45, or parts thereof.

### Methods of Use

The invention also includes methods of using the siRNA molecules and pharmaceutical compositions containing them to prevent or treat Ebola virus disease. A therapeutically effective amount of the composition of the invention is administered to a subject. In one embodiment, the subject is a mammal. In one aspect of this embodiment, the mammal is a laboratory animal, such as a rodent. In another aspect of this embodiment, the mammal is a non-human primate. In still another aspect of this embodiment, the mammal is a human. As used herein, a “therapeutically effective amount” is an amount that prevents, reduces the severity of, or cures Ebola virus disease. Such amounts are determinable by persons skilled in the art, given the teachings contained herein. In one embodiment, a therapeutically effective amount of the pharmaceutical composition administered to a human comprises about 1 mg of the siRNA molecules per kilogram of body weight of the human to about 5 mg of the siRNA molecules per kilogram of body weight of the human. Routes of administration are also determinable by persons skilled in the art, given the teachings contained herein. In one embodiment, the compositions are administered intravenously. In another embodiment, the compositions are administered intranasally.

### DETAILED DESCRIPTION OF THE INVENTION

### Target Selection

Genus Ebolavirus is a member of the Filoviridae family (filovirus). Genus Ebolavirus comprises 5 distinct species: Zaire ebolavirus (EBOV or ZEBOV), Sudan ebolavirus (SUDV), Tai Forest ebolavirus (TAFV), Reston ebolavirus (RESTV), and Bundibugyo ebolavirus (BDBV), where BDBV, EBOV, and SUDV have been associated with large EVD outbreaks in the West Africa. EBOV carries a negative-sense RNA genome in virions that are cylindrical/tubular, and contain viral envelope, matrix, and nucleocapsid components. The overall cylinders are generally approx. 80 nm in diameter, and having a virally encoded glycoprotein (GP) projecting as 7-10 nm long spikes from its lipid bilayer surface. The cylinders are of variable length, typically 800 nm, but sometimes up to 1000 nm long. The outer viral envelope of the virion is derived by budding from domains of host cell membrane into which the GP spikes have been inserted during their biosynthesis. Individual GP molecules appear with spacings of about 10 nm.

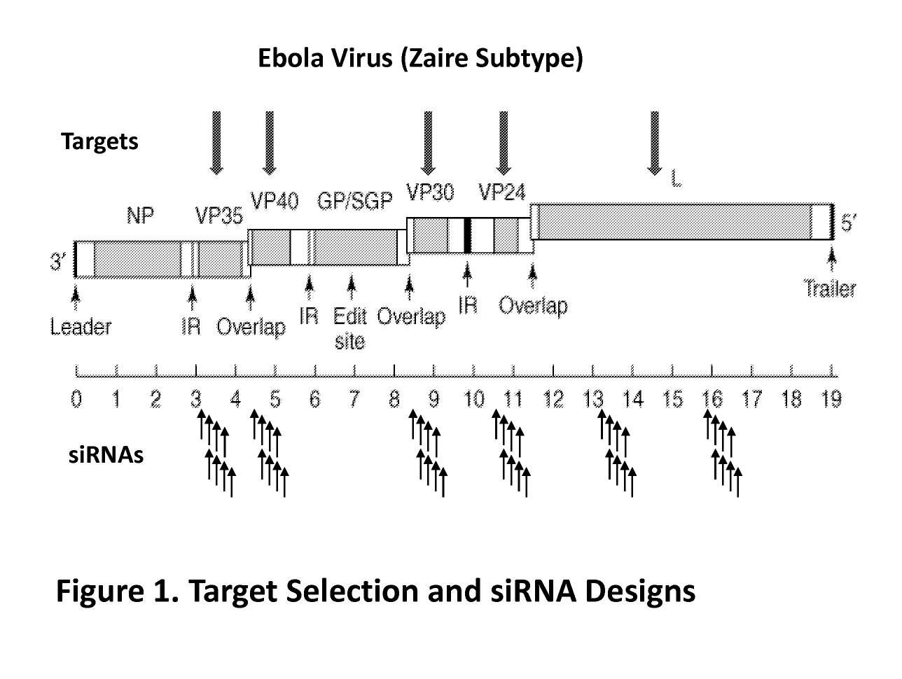

Viral proteins VP40 and VP24 are located between the envelope and the nucleocapsid in the matrix space. At the center of the virion structure is the nucleocapsid, which is composed of a series of viral proteins attached to a 18-19 kb linear, negative-sense RNA without 3′-polyadenylation or 5′-capping; the RNA is helically wound and complexed with the NP, VP35, VP30, and L proteins; this helix has a diameter of 80 nm and contains a central channel of 20-30 nm in diameter (FIG. 1). The overall shape of the virions after purification and visualization (e.g., by ultracentrifugation and electron microscopy, respectively) varies considerably; simple cylinders are far less prevalent than structures showing reversed direction, branches, and loops (i.e., U-, shepherd's crook-, 9- or eye bolt-shapes, or other or circular/coiled appearances), the origin of which may be in the laboratory techniques applied. The characteristic “threadlike” structure is, however, a more general morphologic characteristic of filoviruses (alongside their GP-decorated viral envelope, RNA nucleocapsid, etc.).

Each virion contains one molecule of linear, single-stranded, negative-sense RNA, 18,959 to 18,961 nucleotides in length. The 3′ terminus is not polyadenylated and the 5′ end is not capped. It was found that 472 nucleotides from the 3′ end and 731 nucleotides from the 5′ end are sufficient for replication. It codes for seven structural proteins and one non-structural protein. The gene order is 3′-leader-NP-VP35-VP40-GP/sGP-VP30-VP24-L-trailer-5′; with the leader and trailer being non-transcribed regions, which carry important signals to control transcription, replication, and packaging of the viral genomes into new virions. The genomic material by itself is not infectious, because viral proteins, among them the RNA-dependent RNA polymerase, are necessary to transcribe the viral genome into mRNAs because it is a negative sense RNA virus, as well as for replication of the viral genome. Sections of the NP, VP35 and the L genes from filoviruses have been identified as endogenous in the genomes of several groups of small mammals.

Being acellular, viruses such as Ebola do not replicate through any type of cell division; rather, they use a combination of host- and virally encoded enzymes, alongside host cell structures, to produce multiple copies of themselves; these then self-assemble into viral macromolecular structures in the host cell. The virus completes a set of steps when infecting each individual cell: The virus begins its attack by attaching to host receptors through the glycoprotein (GP) surface peplomer and is endocytosed into macropinosomes in the host cell. To penetrate the cell, the viral membrane fuses with vesicle membrane, and the nucleocapsid is released into the cytoplasm. Encapsidated, negative-sense genomic ssRNA is used as a template for the synthesis (3′-5′) of polyadenylated, monocistronic mRNAs and, using the host cell's ribosomes, tRNA molecules, etc., the mRNA is translated into individual viral proteins.

These viral proteins are processed, a glycoprotein precursor (GP0) is cleaved to GP1 and GP2, which are then heavily glycosylated using cellular enzymes and substrates. These two molecules assemble, first into heterodimers, and then into trimers to give the surface peplomers. Secreted glycoprotein (sGP) precursor is cleaved to sGP and delta peptide, both of which are released from the cell. As viral protein levels rise, a switch occurs from translation to replication. Using the negative-sense genomic RNA as a template, a complementary +ssRNA is synthesized; this is then used as a template for the synthesis of new genomic (−) ssRNA, which is rapidly encapsidated. The newly formed nucleocapsids and envelope proteins associate at the host cell's plasma membrane; budding occurs, destroying the cell.

We selected VP24, VP30, VP35, VP40 and polymerase L of Ebola virus as the targets for an siRNA cocktail-mediated therapeutic approach. The present invention provides siRNA molecules comprising a double-stranded sequence of 17, 18, 19, 20, 21, 22, 23, 24 or 25 nucleotides in length, wherein the siRNA molecules inhibit expression of the VP24, VP30, VP35, VP40 and polymerase L of Ebola virus. The siRNA molecule has blunt ends, or has 3′ overhangs of one or more nucleotides on both sides of the double-stranded region. The siRNA cocktail of the invention contains two, three, four or more sequences set targeting the VP24, VP30, VP35, VP40 and polymerase L of Ebola virus. The siRNA induces specific viral mRNA cleavage by targeting the VP24, VP30, VP35, VP40 and polymerase L of Ebola virus.

### Conserved Viral Genomes

In another aspect, the present invention provides the siRNA molecules targeting the conserved regions of three distinct subtypes of Ebola virus: EBOV, SUDV and BDBV, which caused the large EVD outbreaks in Africa. Complete genome sequences for EBOV are set forth in, e.g., Genbank Accession Nos. AY354458; AF272001; KC242800; KJ660346; and HQ613403. Complete genome sequences for SUDV are set forth in, e.g., Genbank Accession Nos. KC242783; AY729654; EU338380; FJ968794; and KC545389. Complete genome sequences for BDBV are set forth in, e.g., Genbank Accession Nos. KC545393; FJ217161; KC545396; and KC545393. All of these sequences are incorporated herein by reference. The conserved regions of three subtypes encoding the VP24, VP30, VP35, VP40 and polymerase L of Ebola virus have been selected as the sequences of the siRNA molecules. As shown in Table 1, we have aligned all 21 published viral sequences and then designed siRNAs targeting five selected genes.

siRNA Design and Sequence Selection

Using our proprietary bioinformatics programs, we have identified 25 mer blunt ended siRNAs against key regions of genes important to the replication lifecycle of Ebola viruses. These genes included VP24, VP30, VP35, VP40 and polymerase L genes but each and every gene of all Ebola virus strains are included within the invention.

The software predicts siRNAs with appropriate thermodynamic properties, removes known immune stimulating motifs and those siRNAs which were not within the appropriate range for GC content (35-65%). It determines siRNAs of a given length (21 mer with over hangs, or 25 base blunt ended duplex RNAs) that have identity to a gene that we are aiming to silence.

It is well known that the minimum effective length of an siRNA that can silence a gene with high potency is a 19 base duplex, although shorter lengths can have some effectiveness. We have found that a 25 mer is more potent than a 19 mer in silencing a gene in vitro. To ensure optimal thermodynamic properties for the siRNAs, it is important that the first 2 bases do not include a T or A, while it is better that the last 2 bases in the siRNA are a T or A. Without being bound by theory, it is believed that such bases allow the duplex siRNA to unwind more easily and pairing of the antisense strand within Dicer. While a region of identity in many siRNAs can include both a T and an A base at the terminus, it is sometimes not feasible to identify an siRNA with absolute identity in this region. Consequently, if the base pairing in the terminal region is not exact, this may help with efficacy of the siRNA. Therefore, our algorithm for scoring predicted siRNA sequences provides a weighting for siRNAs that meet all of our criteria (exact identity along entire length and have the correct bases at both the 2 start bases and 2 end bases in the sequence). Such a sequence would be weighted according to the formula:

[(Predicted siRNA length−minimum effective siRNA length)*(consecutive base matches)]/number of base mismatches+1. For example, a 25 mer with exact identity would score as follows [(25−19)*(25)]/1)=150.

If we had a 25 mer siRNA with the following sequence CCA TCG TTC CAA GGG TAC GGC ATA A, i.e., 1 base mismatch at the end, the value would look like [(25−19)*(24)]/2)=72. A sequence as follows CCA TCG TTC CAA GGG TAC GTC ATA A would score [(25−19)*(19)]/2)=57, so a base mismatch further from the terminus would reduce the score. Further, a sequence like CCA TCG TTC CAA GGG TAC Gttccgg, where all of the last 6 bases match, would be scored as [(25−19)*(19)]/6)=19. This provides an estimate of how to rank the siRNAs of interest to pursue against a viral strain. Additional weighting can be provided, based on the prevalent disease strain in the general population or even within a strain the isolate that is being observed most frequently.

By expanding the searches we perform against all genes and across all sequenced Ebola strains, we can identify siRNAs that will be able to a) exhibit a broad strain specificity or b) be selective for a specific gene within a particular viral strain. By combining 1 siRNA from the list with broad strain activity (e.g. against VP30) with an siRNA specific for another gene (e.g. VP24 or VP40) from Ebola together with a third siRNA against another gene target in the virus, we can produce a cocktail with broad efficacy against multiple strains of the virus, yet the multi-targeting will prevent the ability of viral mutation to escape therapeutic pressure.

By identifying siRNA sequences against other key target genes within the virus and then looking at overlap between the number of strains that these siRNA cocktails may then target, we have shown that a combination of an siRNA against VP35 with broad strain specificity, when combined with an siRNA against L Polymerase also with broad strain specificity, can increase the potential strains that are targeted by one or both siRNAs. We can see that strain coverage increases to ˜500 fully sequenced Ebola viruses. The degree of coverage for Zaire ebolavirus (EBOV) and Sudan ebolavirus (SUDV) is about equal, suggesting these selected siRNAs may be targeting conserved regions within many of these sequenced strains. We also see coverage of less pathogenic strains, such as Reston ebolavirus (RESTV), with the combined sequences. Such conservation in gene regions may provide a point of vulnerability for the Ebola virus, since these regions may be expected to exhibit lower frequencies of mutation and make them better targets for therapeutic intervention. By multiplexing the therapeutic strategy (delivery of siRNAs against more than one viral gene), we can expect to further limit the potential escape of the virus from therapeutic pressure by mutation in a single gene. The prospect of mutation in two genes covered specifically by siRNA sequences is extremely unlikely.

Even by including these two siRNAs in a cocktail, we may target as many as strains in the BioHealthBase database. The cross-targeting between two virus subtypes means we will provide a single therapeutic against a geographically widespread but less pathogenic Ebola virus with cross reactivity against the less widespread but more lethal Ebola virus.

There are two ways to predict siRNAs against conserved regions of viral genes. One way is to align all the genes from multiple strains and identify overlapping regions within the gene and then determine whether siRNAs with relevant pharmacological properties can be designed against these specific regions. This approach severely limits the number of siRNAs that can be predicted and siRNAs designed with these criteria may exhibit lower potency in gene silencing.

The method we have chosen is to predict siRNAs de novo against each gene sequence within all sequenced viral strains applying the optimal thermodynamic criteria for siRNA potency. This provides a database containing siRNAs that can rapidly be pressed into use in a cocktail of multi-targeted siRNAs. After the siRNA sequences are predicted, we then compare the predicted siRNA sequence across all genes in all viral strains to identify those that exhibit the ability to show identity to and silence the gene in the largest number of species. We then compare the strain coverage that might be expected when we combine siRNAs against select genes into a cocktail targeting three distinct genes. Furthermore, we will design criteria into our selection algorithm that can provide a “weighting” for the most prevalent strain in the general population, which will allow tailoring of a cocktail to aid in treatment of EVD. The ability to rapidly synthesize and characterize siRNAs, together with the fact that an siRNA of the same length demonstrates similar charge and hydrophobicity as well as molecular weight, makes the formulation of such siRNAs very much easier than a vaccine or small molecule. Consequently, a delivery vehicle that shows delivery of one siRNA sequence to the blood stream may be expected to deliver an siRNA against a distinct sequence with equal efficacy. This benefit means that as the predominant Ebola virus changes, the siRNA therapeutic cocktail can also be rapidly tailored to ensure therapeutic benefit to patients.

Currently, our algorithms and predictions have been based on siRNA sequence identity with the gene target within a virus gene. SiRNAs as short as 19 mer have been demonstrated to effectively silence genes in vitro and in vivo. Since many of our siRNAs are 25 bases in length, there may be some sequence redundancy that will still allow complete silencing of a target gene. We are building tools to allow examination of regions that may not be 100% identical to the siRNA sequence across all bases but which show identity in 19 consecutive bases. The ability to identify those genes that do not exhibit complete identity along all 25 bases of the siRNA may increase the number of viral strains that can possibly be targeted by an siRNA. The prediction algorithm provides an increased weighting for genes with identity across all 25 bases, and a weighting of the results based on a change in the expected thermodynamic properties can be made where shorter regions overlap (see above). Such a scoring scale allows increased scores for consecutive regions of identity.

The siRNAs predicted from our algorithms are further evaluated with in vitro assays, and the algorithm is modified to incorporate the empirical results based on efficacy of the siRNAs against viral titer. In this way, possible regions within the viral genome with secondary structure that inhibits the pairing of the siRNA and the gene within DICER can be excluded from consideration for siRNA prediction. As we identify these regions, their sequence may be included in an exclusion algorithm for the siRNAs to be tested. The data obtained from these studies will allow us to predict siRNAs with the greatest scope of viral inhibition to be included in a therapeutic cocktail that can be administered locally or systemically.

We have designed at least sixteen siRNA sequences targeting the conserved regions of each Ebola virus gene selected, among which half of them are 25 mer oligos and the other half are 21 mer oligos (Tables 2, 3, 4, 5, 6). The sequences listed in those tables are positive strands of siRNAs. The 25 mer siRNAs are designed as double-stranded RNA oligos with blunt ends. The 21 mer siRNAs are designed as double-stranded RNA oligos with 3′ end dtdt overhang. Those siRNA oligos will be tested and selected based on their efficacies against Ebola viruses both in vitro and in vivo. We will use the siRNA oligos published from a previous work (Table 7) as the positive control during our cell culture study. The primers we have designed (Table 8) will be used for RT-PCR analyses for detection of the viral mRNA expression levels and viral genomic RNA and complimentary RNA levels.

HKP-Based Polymeric Nanoparticle siRNA Delivery System

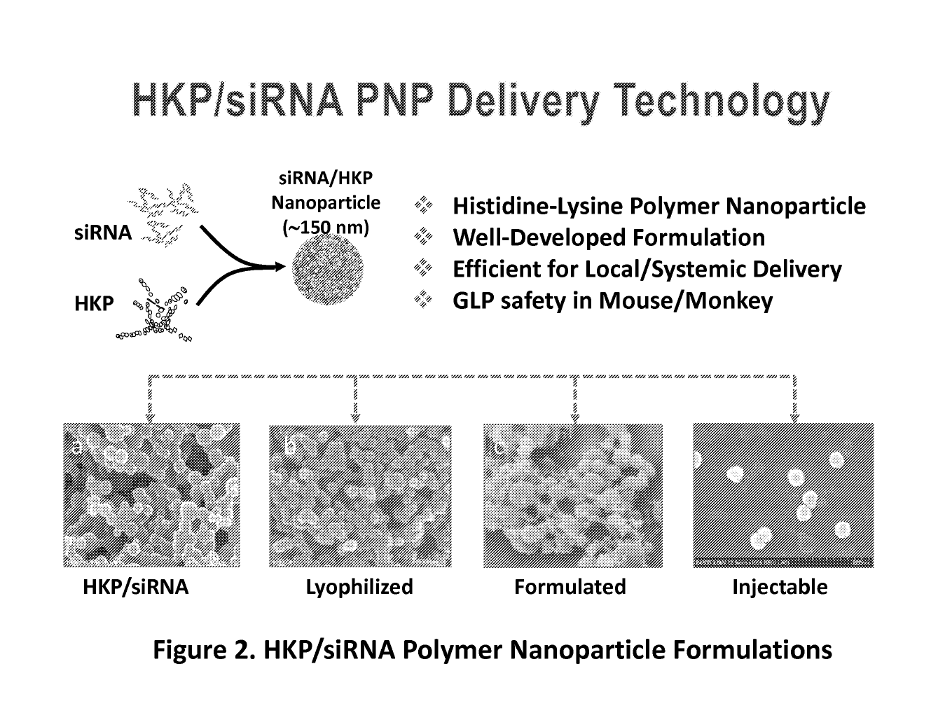

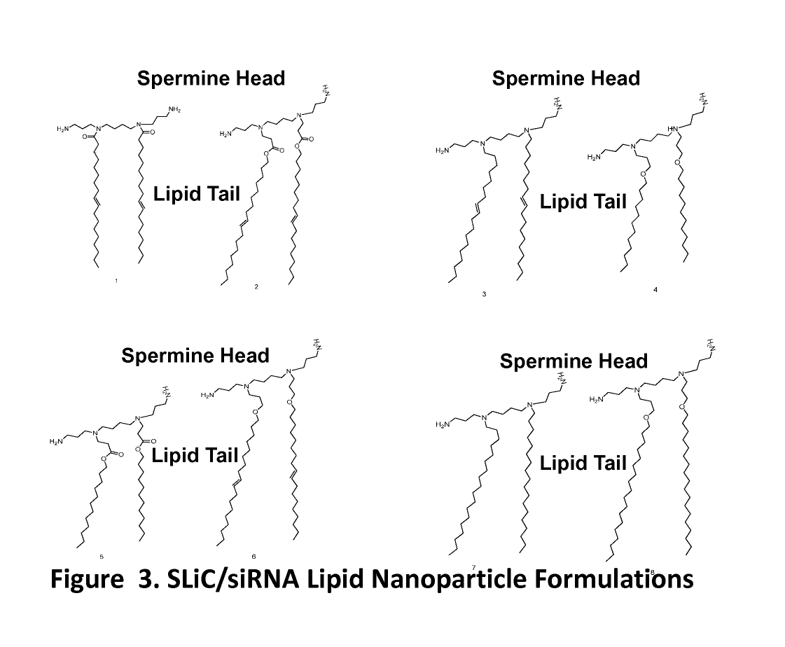

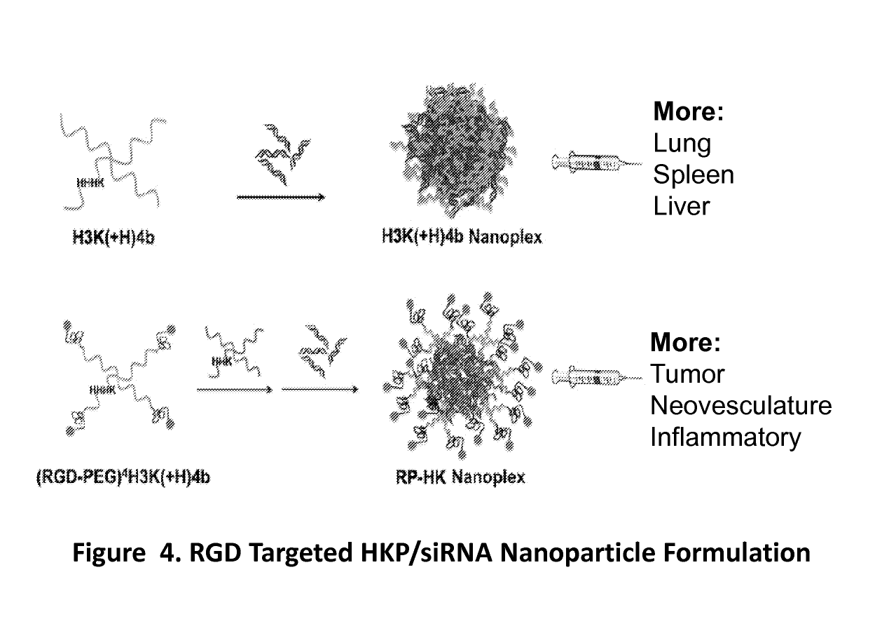

The present invention also provides a therapeutic agent, HKP-siRNA Nanoparticle, including the siRNA molecules and a pharmaceutically acceptable carrier. Histidine-lysine polymers (HKP) have been applied for siRNA deliveries in vitro and in vivo. A pair of the HK polymer species, H3K4b and H3K(+H)4b, has a Lysine backbone with four branches containing multiple repeats of Histidine, Lysine or Asparagine. When this HKP aqueous solution was mixed with siRNA at a N/P ratio of 4:1, or 3:1, or 5:1 by mass, the nanoparticles (average size of 150 nm in diameter) were self-assembled (FIG. 2). Optimal branched histidine-lysine polymer, HKP, was synthesized on a Ranin Voyager synthesizer (PTI, Tucson, Ariz.). The two species of the HKP used in the study were H3K4b with a structure of (R)K(R)-K(R)-(R)K(X), for H3K4b where R=KHHHKHHHKHHHKHHHK; K=lysine and H=histidine. The particle size and zeta-potential were measured with Blookheaven's Particle Sizer 90 Plus (FIG. 3). The HKP-siRNA aqueous solution was semi-transparent without noticeable aggregation of precipitate, and can be stored at 4° C. for at least three months. We have also developed a process for lyophilizing this HKP-siRNA solution into dry powder (FIG. 4). After dissolving this dry powder with PBS or D5W, the therapeutic agent can be administrated into the blood stream through IV infusion.

SLiC-Based Lipid Nanoparticle siRNA Delivery System

We also developed a novel drug delivery carrier comprised of a new cationic lipid and cholesterol. The cationic lipid is made of a spermine head and an oleyl alcohol tail which is conjugated with the cholesterol as a novel and unique siRNA delivery system (Spermine-Lipid Cholesterol, SLiC). The CAS Registry Number of spermine is 71-44-3. The information provided by this CAS entry is incorporated herein by reference in its entirety. When SLiC is mixed with siRNA duplexes in aqueous solution, siRNA/SLiC nanoparticles are formulated through a self-assembling process. This new form of lipid nanoparticle is readily biodegradable. FIG. 3 shows several species of this novel lipid structure with spermine as cationic head and oleyl alcohol tails conjugated through various bonds at two tertiary amine groups in the middle. We have applied this carrier for siRNA transfection in Hela cells and 293 cells for target gene knockdown in vitro, and we also tested it with mouse model for in vivo delivery of siRNA.

RGD-Mediated Endothelial Cell Targeting for EVD siRNA Therapeutics

The carrier comprises at least one of the following: a Histidine-Lysine (HK) polymer to deliver the siRNA molecule into a cell of an animal or a human being. The carriers not only provide the protection of nucleic acids from degradation in the cell, but also facilitate the entry of the siRNA into the cytoplasm. In turn, the siRNA induce the decay of the viral gene expression and inhibit the viral replication in the cytoplasm. The HK polymers were proved to increase the delivery efficiency of the siRNA molecules in vitro and in vivo using as the carrier. The carrier can also comprise peptide ligands containing the Arginine-Glycine-Aspartate (RGD) domain, such as H-ACRGDMFGCA-OH. The RGD peptide ligands display a strong affinity and selectivity to the αvβ3 and αvβ5 integrins on the cell surface. The integrins are transmembrane receptors which are associated to the cell-cell and cell-extracellular matrix (CEM) interaction. The αvβ3 integrins are mainly distributed on the surface of activated endothelial cells, melanoma, and glioblastoma; and the αvβ5 integrins are widely distributed on the surface of mammalian cells, especially on the epithelial cells, fibroblasts and platelets.

Ebola virus transmits between humans from direct contact of the bodily fluids of the infected human. Ebola virus is the most active in infecting fibroblasts under the skin and in the lymph nodes. This allow the virus quickly enter the blood and damage the lymphocyte homing at high endothelial venules (HEV). The epithelial cells may also be infected when the newly generated virus are released from infected fibroblasts and mononuclear cells. Thus, a carrier that includes an RGD peptide ligand may deliver the therapeutic siRNA molecules to the important cells types during Ebola virus infection, such as fibroblasts, epithelial cells, endothelial cells and platelets by targeting the αvβ3 and αvβ5 integrins on the surface of those cells with high affinity and specificity, and sequentially inhibit the viral gene expression inside those infected cells. Both T-cell immunoglobulin and mucin domain 1 (TIM-1) (also known as Hepatitis A virus cellular receptor 1 (HAVcr-1)) and Niemann-Pick C1 (NPC1) have been identified to be the host receptors binding to the glycoprotein (GP) of the Ebola virus and mediating the virus cellular entry. TIM-1 specific antibody ARDS inhibited the Ebola virus infectivity in vitro. In an animal study, the mice that were heterozygous for NPC1 were protected from lethal Ebola virus challenge. TIM-1 distributes on the surface of human mucosal epithelial cells, such as trachea, cornea and conjunctiva. NPC1 is a putative integral membrane protein which plays a critical role in the intracellular lipid transport.

### Testing

The selected siRNA molecules and pharmaceutical compositions are tested in vitro and in vivo, for example, in a cell culture or in an animal model. In one embodiment, the cell culture comprises a Vero E6 cell culture. In another embodiment, the animal model comprises an Ebola virus infected guinea pig model. In still another embodiment, the animal model comprises an Ebola virus infected non-human primate model. In a further embodiment, anti-Ebola virus activity is measured with viral gene silencing using RT-PCR, Race Analysis, Northern Blot, or Western Blot.

### EXAMPLES

### Example 1. Selection of siRNA Cocktails for EVD Treatment

One important concept for designing siRNA therapeutic against viral infection is to use an siRNA cocktail with multiple siRNA oligos targeting multiple viral genes at the same time. Due to the unique function of each of those viral genes for viral proliferation and infection, and their potential function blocking RNAi machinery, we will target three viral genes at the same time. Cocktail No. 1 (CT01) is targeting VP24 and VP35 and LP protein; Cocktail No. 2 (CT02) is targeting VP30 and VP35 and LP protein; Cocktail No. 3 (CT03) is targeting VP24 and VP40 and LP protein; Cocktail No. 4 (CT04) is targeting VP30 and VP40 and LP protein; Cocktail No. 5 (CT05) is targeting VP35 and VP40 and LP protein; Cocktail No. 6 (CT06) is targeting VP24 and VP30 and VP35; Cocktail No. 7 (CT07) is targeting VP30 and VP35 and VP40; Cocktail No. 8 (CT08) is targeting VP24 and VP30 and LP protein.

### Example 2. Sequences of the siRNA Cocktail CT01

CT01 (25) contains:
VP24 (3):
5′-guggaagguuuauugggcugguauu-3′,
VP35(4):
5′-cuucauuggcuacuguugtgcaaca-3′,
and
LP (5):
5′-cauuaaguacacaaugcaagaugcu-3′.
CT01 (21) contains:
VP24 (9):
5′-ggacgauacaaucuaauaudtdt-3′,
VP35 (9):
5′-gagcagcuaaugaccggaadtdt-3′,
and
LP (9):
5′-gaccaaugugaccuugucadtdt-3′.

### Example 3. Sequences of the siRNA Cocktail CT02

CT02 (25) contains:
VP30 (3):
5′-ggagaguuuaacugauagggaauua-3′;
VP35 (4)
5′-cuucauuggcuacuguugtgcaaca-3′,
and
LP (5):
5′-cauuaaguacacaaugcaagaugcu-3′.
CT02 (21) contains:
VP30 (9):
5′-ucgaggugaguaccgucaadtdt-3′,
and
VP35 (9):
5′-gagcagcuaaugaccggaadtdt-3′,
and
LP (9):
5′-gaccaaugugaccuugucadtdt-3′.

### Example 4. Sequences of the siRNA Cocktail CT03

CT03 (25) contains:
VP24 (3):
5′-guggaagguuuauugggcugguauu-3′,
and
VP40 (3)
5′-cuccaucaaauccacucagaccaau-3′,
and
LP (5):
5′-cauuaaguacacaaugcaagaugcu-3′.
CT03 (21) contains:
VP24 (9):
5′-ggacgauacaaucuaauaudtdt-3′,
VP40 (9):
5′-gguuauauuaccuacugcudtdt-3′,
and
LP (9):
5′-gaccaaugugaccuugucadtdt-3′.

### Example 5. Sequences of the siRNA Cocktail CT04

CT04 (25) contains:
VP30 (3):
5′-ggagaguuuaacugauagggaauua-3′,
and
VP40 (3):
5′-cuccaucaaauccacucagaccaau-3′,
and
LP (5):
5′-cauuaaguacacaaugcaagaugcu-3′.
CT04 (21):
VP30 (9):
5′-ucgaggugaguaccgucaadtdt-3′,
and
VP40 (9):
5′-gguuauauuaccuacugcudtdt-3′,
and
LP (9):
5′-gaccaaugugaccuugucadtdt-3′.

### Example 6. Sequences of the siRNA Cocktail CT05

CT05 (25) contains:
VP35 (4)
5′-cuucauuggcuacuguugtgcaaca-3′,
VP40 (3):
5′-cuccaucaaauccacucagaccaau-3′,
and
LP (5):
5′-cauuaaguacacaaugcaagaugcu-3′.
CT05 (21) contains:
VP35 (9):
5′-gagcagcuaaugaccggaadtdt-3′,
and
VP40 (9):
5′-gguuauauuaccuacugcudtdt-3′,
and
LP (9):
5′-gaccaaugugaccuugucadtdt-3′.

### Example 7. Sequences of the siRNA Cocktail CT06

CT06 (25) contains:
VP24 (3):
5′-guggaagguuuauugggcugguauu-3′,
and
VP30 (3):
5′-ggagaguuuaacugauagggaauua-3′,
and
VP35 (4)
5′-cuucauuggcuacuguugtgcaaca-3′.
CT06 (21) contains:
VP24 (9):
5′-ggacgauacaaucuaauaudtdt-3′,
and
VP30 (9):
5′-ucgaggugaguaccgucaadtdt-3′,
and
VP35 (9):
5′-gagcagcuaaugaccggaadtdt-3′.

### Example 8. Sequences of the siRNA Cocktail CT07

CT07 (25) contains:
VP30 (3):
5′-ggagaguuuaacugauagggaauua-3′,
and
VP35 (4)
5′-cuucauuggcuacuguugtgcaaca-3′,
and
VP40 (3):
5′-cuccaucaaauccacucagaccaau-3′.
CT07 (21) contains:
VP30 (9):
5′-ucgaggugaguaccgucaadtdt-3′,
and
VP35 (9):
5′-gagcagcuaaugaccggaadtdt-3′,
VP40 (9):
5′-gguuauauuaccuacugcudtdt-3′.

### Example 9. Sequences of the siRNA Cocktail CT08

CT08 (25) contains:
VP24 (3):
5′-guggaagguuuauugggcugguauu-3′,
and
VP30 (3):
5′-ggagaguuuaacugauagggaauua-3′,
and
LP (5):
5′-cauuaaguacacaaugcaagaugcu-3′.
CT08 (21) contains:
VP24 (9):
5′-ggacgauacaaucuaauaudtdt-3′,
and
VP30 (9):
5′-ucgaggugaguaccgucaadtdt-3′,
and
LP (9):
5′-gaccaaugugaccuugucadtdt-3′.

### Example 10. VP24 siRNA is Paired with Other Four siRNA Sequences and Each Pair of siRNA Duplexes Will be Used as One Drug Component

PA01 (25) = (VP24-VP30) siRNAs:
VP24 (5):
5′-gcauggucaaugacaaggaaucucu-3′
and
VP30 (5):
5′-cuuguugacucugaucaagacggca-3′.
PA01 (21) = (VP24-VP30) siRNAs:
VP24 (13):
5′-ccucgacacgaaugcaaagdtdt-3′
and
VP30 (13):
5′-gacucugaucaagacggcadtdt-3′.
PA02 (25) = (VP24-VP35) siRNAs:
VP24 (5):
5′-gcauggucaaugacaaggaaucucu-3′
and
VP35 (5)
5′-cgaauagcaaaccuugaggccagcu-3′.
PA02 (21) = (VP24-VP35) siRNAs:
VP24 (13):
5′-ccucgacacgaaugcaaagdtdt-3′
and
VP35 (13):
5′-gcaaaccuugaggccagcudtdt-3′.
PA03 (25) = (VP24-VP40) siRNAs:
VP24 (5):
5′-gcauggucaaugacaaggaaucucu-3′
and
VP40 (5)
5′-cagcauucauccuugaagcuauggu-3′.
PA03 (21) = (VP24-VP40) siRNAs:
VP24 (13):
5′-ccucgacacgaaugcaaagdtdt-3′
and
VP40 (13):
5′-cauucauccuugaagcuaudtdt-3′.
PA04 (25) = (VP24-LP) siRNAs:
VP24 (5):
5′-gcauggucaaugacaaggaaucucu-3′
and
LP (5):
5′-cauuaaguacacaaugcaagaugcu-3′.
PA04 (21) = (VP24-LP) siRNAs:
VP24 (13):
5′-ccucgacacgaaugcaaagdtdt-3′
and
LP (13):
5′-cauuaaguacacaaugcaadtdt-3′.

### Example 11. VP30 siRNA Paired with Other Three siRNA Sequences and Each Pair of siRNA Duplexes Will be Used as One Drug Component

PA05 (25) = (VP30-VP35) siRNAs:
VP30 (5):
5′-cuuguugacucugaucaagacggca-3′
and
VP35 (5):
5′-cgaauagcaaaccuugaggccagcu-3′.
PA05 (21) = (VP30-VP35) siRNAs:
VP30 (13):
5′-gacucugaucaagacggcadtdt-3′
and
VP35 (13):
5′-gcaaaccuugaggccagcudtdt-3′.
PA06 (25) = (VP30-VP40) siRNAs:
VP30 (5):
5′-cuuguugacucugaucaagacggca-3′
and
VP40 (5):
5′-cagcauucauccuugaagcuauggu-3′.
PA06 (21) = (VP30-VP40) siRNAs:
VP30 (13):
5′-gacucugaucaagacggcadtdt-3′
and
VP40 (13):
5′-cauucauccuugaagcuaudtdt-3′.
PA07 (25) = (VP30-LP) siRNAs:
VP30 (5):
5′-cuuguugacucugaucaagacggca-3′
and
LP (5):
5′-cauuaaguacacaaugcaagaugcu-3′.
PA07 (21) = (VP30-LP) siRNAs:
VP30 (13):
5′-gacucugaucaagacggcadtdt-3′
and
LP (13):
5′-cauuaaguacacaaugcaadtdt-3′.

### Example 12. VP35 siRNA Paired with Other Two siRNA Sequences and Each Pair of siRNA Duplexes Will be Used as One Drug Component

PA08 (25) = (VP35-VP40) siRNAs:
VP35 (5):
5′-cgaauagcaaaccuugaggccagcu-3′
and
VP40 (5):
5′-cagcauucauccuugaagcuauggu-3′.
PA08 (21) = (VP35-VP40) siRNAs:
VP35 (13):
5′-gcaaaccuugaggccagcudtdt-3′
and
VP40 (13):
5′-cauucauccuugaagcuaudtdt-3′.
PA09 (25) = (VP35-LP) siRNAs:
VP35 (5):
5′-cgaauagcaaaccuugaggccagcu-3′
and
LP (5):
5′-cauuaaguacacaaugcaagaugcu-3′.
PA09 (21) = (VP35-LP) siRNAs:
VP35 (13):
5′-gcaaaccuugaggccagcudtdt-3′
and
LP (13):
5′-cauuaaguacacaaugcaadtdt-3′.

### Example 13. VP40 siRNA Paired with LP siRNA Sequence as One Drug Component

PA10 (25) = (VP40-LP) siRNAs:
VP40 (5):
5′-cagcauucauccuugaagcuauggu-3′
and
LP (5):
5′-cauuaaguacacaaugcaagaugcu-3′.
PA10 (21) = (VP40-LP) siRNAs:
VP40 (13):
5′-cauucauccuugaagcuaudtdt-3′
and
LP (13):
5′-cauuaaguacacaaugcaadtdt-3′.

### Example 14. Sequences of the Primers for RT-PCR Detection of Target Genes

We have designed DNA primers for detection of VP24, VP30, VP35, VP40 and LP protein using RT-PCR analysis. The average size of PCR generated fragments is in the range between 250 to 310 base pair (Table 8).

### Example 15. Cell Culture Based Screening for Potent Anti-Ebola siRNA Oligos

The psiCheck plasmid carrying Ebola viral sequence is used to test the selected siRNA for their potential activity silencing targeted viral gene with Hela cell culture studies. In addition, Vero E6 cell infected with Ebola virus is used to test the selected siRNA for their anti-Ebola viral infection activity.

A. Subcloning Ebola Virus Gene Fragments as Surrogates for siRNA Potency Examination in Vero E6 Cells

In order to test the effects of siRNA candidates on degrading the target genes of Ebola virus, a dual luciferase reporter vector, psiCHECK-2, carrying gene fragment of either VP24, or VP30, or VP35, or VP40 or polymerase L, is designed to provide a quantitative and rapid approach for initial optimization of RNA interference (RNAi) activity. The vectors enable monitoring of changes in expression of a target gene fused to a reporter gene. The DNA fragment of either VP24, or VP30, or VP35, or VP40 or polymerase L is amplified by PCR with specific primers, and then cloned into the multiple cloning sites of psiCHECK-2 Vector. In this vector, Renilla Luciferase is used as a primary reporter gene. The siRNA targeting gene fragments located downstream of the Renilla translational stop codon can be down regulated by the specific siRNA inhibitor and results in decrease of Luciferase expression.

The reporter plasmids (as described above) psi-VP24, psi-VP30, psi-VP35, psi-VP40 and psi-L, and the siRNA candidates can be co-transfected into Vero E6 cells using Lipofectamine 2000 in the DMEM without FBS. The blank psi vector serves as the negative control. 6 h post-transfection. The activity of the firefly luminescence and Renilla Luciferase in which each well can be detected using the Dual Luciferase Kit. Down regulation of the luciferase activities by the siRNA inhibitors indicates that these siRNAs could greatly inhibit the expression of the target genes of Ebola virus when we use them in a Ebola virus infected cell culture model and animal model.

B. Infection of Vero E6 Cells with Ebola Virus

To assess whether the real Ebola viral mRNAs for VP24, VP30, VP35, VP40 and polymerase L can be directly degraded by the selected siRNA inhibitors. The Vero E6 cells infected with EBOV in Biosafety Level-4 laboratory environment can be transfected with the selected single siRNA, or siRNA cocktail or positive/negative control siRNA using Lipofectamine 2000 in the DMEM without FBS, at 6, 12 or 24 h after infection. The cell then can be harvested for quantitative real-time RT-PCR and for RNA isolation followed by a 5′-rapid amplification of cDNA ends (5′-RACE). The viral RNA extracted from the cell supernatants for RT-PCR can be carried out with forward and reverse primers and a TaqMan probe specific to the EBOV glycoprotein. Absolute quantification of viral gene expression levels can be compared with those of the positive or negative control. RNA from the harvested cells can be extracted for 5′-RACE assay with gene-specific primers for cDNA products of VP24, VP30, VP35, VP40 and polymerase L. The length of the PCR products can be used for comparison of the potencies of the selected siRNA inhibitors. The single siRNA or siRNA cocktail with high protection efficiency can be selected for the animal model studies.

### Example 16. Guinea Pig Model Study

The guinea pig model can be used to investigate the efficiency of the siRNA on inhibiting the Ebola infection in vivo. The siRNA cocktail encapsidated with RGD-PEG-HKP tripartite nanoparticle system can be further tested in the animal model, such as the guinea pig system. We can treat eight guinea pigs via intraperitoneal (ip) injection of nanoparticles containing siRNA cocktail and another eight guinea pigs treated with tripartite nanoparticle without siRNA as the control. Three hours after treatment, guinea pigs should be challenged via subcutaneous (sc) injection of EBOV. All guinea pigs should receive 3 additional treatments at 24, 48, 96 h post challenge and should be carefully monitored for signs of disease and survival during the 21-day course of the experiment. The blood can be collected at 1, 4, 7, 10 and 18-day post challenge, and the virus titration can be performed by plaque assay on Vero E6 cells. Three guinea pigs from the treatment and control groups can be euthanized at 24, 72 and 120 h post challenge, respectively. Whole blood sample is collected by cardiac puncture for hematology counts and serum biochemical assays before the guinea pigs were euthanized. The organs including kidney, liver, spleen, pancreas and lung are collected and homogenized, and the virus titration is performed by plaque assay on Vero E6 cells.

The tissues, including kidney, liver, spleen, pancreas, lung, thymus, stomach, small intestine, colon, submandibular salivary gland, brain, uterus and mandibular lymph nodes, are collected and fixed for histopathology and immunohistochemistry.

### Example 17. Non-human Primate Study

A non-human primate study will be performed for investigation of the anti-Ebola efficacy of the siRNA inhibitors using a non-human primate (Chinese rhesus macaques) model challenged with Ebola virus. The study is carried out in Biosafety Level-4 lab. The siRNA/RGD-PEG-HKP tripartite nanoparticle system is an excellent system for evaluation of siRNA drug activity. In the first group, three out of four rhesus macaques are treated with the anti-Ebola siRNA cocktail treatment at 30 min post challenge. The control animal is treated with tripartite nanoparticle without siRNA. The other three animals are tested with additional treatments at 1, 3, and 5-day post challenge, while the control animal is only treated with tripartite nanoparticle without siRNA. In the second group, we will treat the animal with additional dosages every day to investigate if increasing frequency of the treatment could increase the protective efficiency. Three out of four rhesus macaques are treated with the anti-Ebola siRNA cocktail at 30 min post challenge, and then receive six additional treatments at 1, 2, 3, 4, 5 and 6-day post challenge; while the control animal is treated with tripartite nanoparticle without siRNA at the same time points.

The intravenous infusion of saline is carried out with a basic single syringe infusion pump during the course of the entire study. All animals are carefully monitored for signs of disease and survival during the 43-day course of the experiment. The blood samples are collected at 3, 6, 10, 14, 22, and 40-43 day post challenge. The virus titration performed with plaque assay using Vero E6 cells is carried out using the blood samples collected from the rhesus monkeys.

### Tables

Table 1. Ebola Virus Genome Sequences Used for siRNA Design

Total of 21 Ebola viral sequences representing all published Ebola viral sequences were lined up for siRNA design against the conserved sequences within VP24, VP30, VP35, VP40 and LP genes.

Table 2. siRNA Against Ebola Virus Protein 24

The siRNA sequences with either 25 mer blunt-end (×8) or 21 mer dtdt overhang (×8) structure, targeting conserved region of Ebola viral sequences of VP24 gene.

Table 3. siRNA Against Ebola Virus Protein 30

The siRNA sequences with either 25 mer blunt-end (×8) or 21 mer dtdt overhang (×8) structure, targeting conserved region of Ebola viral sequences of VP30 gene.

Table 4. siRNA Against Ebola Virus Protein 35

The siRNA sequences with either 25 mer blunt-end (×8) or 21 mer dtdt overhang (×8) structure, targeting conserved region of Ebola viral sequences of VP35 gene.

Table 5. siRNA Against Ebola Virus Protein 40

The siRNA sequences with either 25 mer blunt-end (×8) or 21 mer dtdt overhang (×8) structure, targeting conserved region of Ebola viral sequences of VP40 gene.

Table 6. siRNA Against Ebola Virus Protein L Polymerase

The siRNA sequences with either 25 mer blunt-end (×8) or 21 mer dtdt overhang (×8) structure, targeting conserved region of Ebola viral sequences of L Polymerase gene.

Table 7. siRNA Duplexes with Demonstrated Potent Anti-Ebola Virus Activity

The siRNA sequences targeting Zaire Ebola Virus have been tested in cell culture model for anti-viral activity. The positive siRNA sequences can be used as the positive control for the in vitro screening experiment.

Table 8. DNA Primers for RT-PCR Analysis of Viral RNA Levels

The primers for RT-PCR analysis have been designed and selected for detection of viral sequences to measure expression levels of viral gene after treatment of the viral infected cell.

### REFERENCES

1. Choi J H, Croyle M A. Emerging targets and novel approaches to Ebola virus prophylaxis and treatment. BioDrugs. 2013 December; 27(6):565-83.
2. Johnson, Karl M.; Kawaoka, Yoshihiro; Lipkin, W. Ian; Negredo, Ana I et al. (2010). “Proposal for a revised taxonomy of the family Filoviridae: Classification, names of taxa and viruses, and virus abbreviations”. Archives of Virology 155 (12): 2083-103.
3. Choi J H, Croyle M A. Emerging targets and novel approaches to Ebola virus prophylaxis and treatment. BioDrugs. 2013. 27(6):565-83.
4. Lu J, et al. Host IQGAP1 and Ebola virus VP40 interactions facilitate virus-like particle egress. J Virol. 2013 July; 87(13):7777-80.
5. Geisbert T W, et al. Postexposure protection of non-human primates against a lethal Ebola virus challenge with RNA interference: a proof-of-concept study. Lancet. 2010. 375(9729):1896-905.
6. Geisbert T W, et al. Postexposure protection of guinea pigs against a lethal ebola virus challenge is conferred by RNA interference. J Infect Dis. 2006. 15; 193(12):1650-7.
7. Thi E P, et al. Marburg virus infection in nonhuman primates: Therapeutic treatment by lipid-encapsulated siRNA. Sci Transl Med. 2014. 20; 6(250):
8. Zhu Y, et al. Characterization of the RNA silencing suppression activity of the Ebola virus VP35 protein in plants and mammalian cells. J Virol. 2012. 86(6):3038-49.
9. Mateo M, et al. Knockdown of Ebola virus VP24 impairs viral nucleocapsid assembly and prevents virus replication. J Infect Dis. 2011. 204 Suppl 3:S892-6.
10. Fabozzi G, et al. Ebolavirus proteins suppress the effects of small interfering RNA by direct interaction with the mammalian RNAinterference pathway. J Virol. 2011. 85(6):2512-23.
11. Spurgers K B, et al. Identification of essential filovirion-associated host factors by serial proteomic analysis and RNAi screen. Mol Cell Proteomics. 2010. (12):2690-703.
12. Kolokoltsov A A, et al. Identification of novel cellular targets for therapeutic intervention against Ebola virus infection by siRNAscreening. Drug Dev Res. 2009. 70(4):255-265.
13. Pattyn, S.; Jacob, W.; van der Groen, G.; Piot, P.; Courteille, G. (1977). “Isolation of Marburg-like virus from a case of haemorrhagic fever in Zaire”. Lancet 309 (8011): 573-4.
14. Bowen, E. T. W.; Lloyd, G.; Harris, W. J.; Platt, G. S.; Baskerville, A.; Vella, E. E. (1977). “Viral haemorrhagic fever in southern Sudan and northern Zaire. Preliminary studies on the aetiological agent”. Lancet 309 (8011): 571-3.
15. WHO. “Ebola virus disease”.
16. Nanbo, Asuka; Watanabe, Shinji; Halfmann, Peter; Kawaoka, Yoshihiro (4 Feb. 2013). “The spatio-temporal distribution dynamics of Ebola virus proteins and RNA in infected cells”. Nature. doi:10.1038/srep01206.
17. Feldmann, H. K. (1993). “Molecular biology and evolution of filoviruses”. Archives of virology. Supplementum 7: 81-100.
18. Klenk, H-D; Feldmann, H (editor) (2004). Ebola and Marburg Viruses: Molecular and Cellular Biology. Horizon Bioscience. ISBN 978-1-904933-49-6. Taylor, D.; Leach, R.; Bruenn, J. (2010). “Filoviruses are ancient and integrated into mammalian genomes”. BMC Evolutionary Biology 10: 193.
19. Belyi, V. A.; Levine, A. J.; Skalka, A. M. (2010). “Unexpected Inheritance: Multiple Integrations of Ancient Bornavirus and Ebolavirus/Marburgvirus Sequences in Vertebrate Genomes”. In Buchmeier, Michael J. PLoS Pathogens 6 (7): e1001030.
20. Taylor, D. J. et al. (2014). “Evidence that ebolaviruses and cuevaviruses have been diverging from marburgviruses since the Miocene”. PeerJ 2: e556.
21. Carette J E, et al. (September 2011). “Ebola virus entry requires the cholesterol transporter Niemann-Pick C1”. Nature 477 (7364): 340-3.
22. Cote M, et al. (September 2011). “Small molecule inhibitors reveal Niemann-Pick C1 is essential for Ebola virus infection”. Nature 477 (7364): 344-8.
23. Flemming A (October 2011). “Achilles heel of Ebola viral entry”. Nat Rev Drug Discov 10(10): 731.
24. Miller E H, et al. (March 2012). “Ebola virus entry requires the host-programmed recognition of an intracellular receptor”. EMBO Journal 31 (8): 1947-60.
25. Kondratowicz A S, Lennemann N J, Sinn P L, et al. (May 2011). “T-cell immunoglobulin and mucin domain 1 (TIM-1) is a receptor for Zaire Ebolavirus and Lake Victoria Marburgvirus”. Proc. Natl. Acad. Sci. U.S.A. 108 (20): 8426-31.
26. Saeed, M. F.; Kolokoltsov, A. A.; Albrecht, T.; Davey, R. A. (2010). “Cellular Entry of Ebola Virus Involves Uptake by a Macropinocytosis-Like Mechanism and Subsequent Trafficking through Early and Late Endosomes”. In Basler, Christopher F. PLoS Pathogens 6 (9): e1001110.
27. Feldmann H (May 2014). “Ebola—A Growing Threat?”. N. Engl. J. Med.doi:10. 1056/NEJMp1405314.
28. Isaacson, M; Sureau, P; Courteille, G; Pattyn, S R; Clinical Aspects of Ebola Virus Disease at the Ngaliema Hospital, Kinshasa, Zaire, 1976. Retrieved 2014-06-24.
29. Brown, Rob (18 Jul. 2014) The virus detective who discovered Ebola in 1976 BBC News Magazine, Retrieved 18 Jul. 2014.
30. Johnson, K. M.; Webb, P. A.; Lange, J. V.; Murphy, F. A. (1977). “Isolation and partial characterisation of a new virus causing haemorrhagic fever in Zambia”. Lancet 309 (8011): 569-71 . . . .
31. Netesov, S. V. et al. (2000). “Family Filoviridae”. In van Regenmortel, M. H. V.; Fauquet, C. M.; Bishop, D. H. L.; Carstens, E. B.; Estes, M. K.; Lemon, S. M.; Maniloff, J.; Mayo, M. A.; McGeoch, D. J. Virus Taxonomy—Seventh Report of the International Committee on Taxonomy of Viruses. San Diego, USA: Academic Press. pp. 539-48.
32. Pringle, C. R. (1998). “Virus taxonomy-San Diego 1998”. Archives of Virology 143 (7): 1449-59.
33. Feldmann, H.; et al. (2005). “Family Filoviridae”. In Fauquet, C. M.; Mayo, M. A.; Maniloff, J.; Desselberger, U.; Ball, L. A. Virus Taxonomy—Eighth Report of the International Committee on Taxonomy of Viruses. San Diego, USA: Elsevier/Academic Press. pp. 645-653.
34. Mayo, M. A. (2002). “ICTV at the Paris ICV: results of the plenary session and the binomial ballot”. Archives of Virology 147 (11): 2254-60.
35. Netesov, S. V.; et al. (2000). “Family Filoviridae”. In van Regenmortel, M. H. V.; Fauquet, C. M.; Bishop, D. H. L.; Carstens, E. B.; Estes, M. K.; Lemon, S. M.; Maniloff, J.; Mayo, M. A.; McGeoch, D. J.; Pringle, C. R.; Wickner, R. B. Virus Taxonomy—Seventh Report of the International Committee on Taxonomy of Viruses. San Diego, USA: Academic Press. pp. 539-48.
36. Pringle, C. R. (1998). “Virus taxonomy-San Diego 1998”. Archives of Virology 143 (7): 1449-59.

All publications identified herein, including issued patents and published patent applications, and all database entries identified herein by url addresses or accession numbers are incorporated herein by reference in their entirety.

Although this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

TABLE 1
Ebola Virus Genome Sequences Used for siRNA Design

| | Accession | Virus | Strain | Location | Date |
|---|---|---|---|---|---|
| 1 | AY354458 | EBOV | Zaire | Kikwit, DRC | 1995 |
| 2 | KC545393 | BDBV | EboBund-112 | Isiro, DRC | 2012 |
| 3 | AF272001 | EBOV | Mayinga | Yambuku, DRC | 1976 |
| 4 | KC242801 | EBOV | deRoover | DRC | 1976 |
| 5 | KC242800 | EBOV | Ilembe | Elombe, Gabon | 2002 |
| 6 | KC242784 | EBOV | Luebo9 | Luebo, DRC | 2007 |
| 7 | KJ660346 | EBOV | Kissidougou-C15 | Kissidougou, Guinea | 2014 |
| 8 | KJ660347 | EBOV | Gueckedou-C07 | Gueckedou, Guinea | 2014 |
| 9 | JQ352763 | EBOV | Kikwit | Kikwit, DRC | 1995/5/4 |
| 10 | HQ613403 | EBOV | M-M | DRC | 2007/8/31 |
| 11 | HQ613402 | EBOV | 034-KS | DRC | 2008/12/31 |
| 12 | KC242783 | SUDV | Maleo | Maleo, Sudan | 1979 |
| 13 | AY729654 | SUDV | Gulu | Gulu, Uganda | 2000 |
| 14 | EU338380 | SUDV | Yambio | Yambio, South Sudan | 2004 |
| 15 | FJ968794 | SUDV | Boniface | Sudan | 1976 |
| 16 | KC545389 | SUDV | EboSud-602 | Kibaale, Uganda | 2012 |
| 17 | KC589025 | SUDV | EboSud-639 | Luwero, Uganda | 22012 |
| 18 | JN638998 | SUDV | Nakisamata | Nakisimata, Uganda | 2011/5/1 |
| 19 | FJ217161 | BDBV | Bundibugyo | Bundibugyo, Uganda | 2007/11/1 |
| 20 | KC545396 | BDBV | EboBund-14 | Isiro, DRC | 2012 |
| 21 | KC545393 | BDBV | EboBund-112 | Isiro, DRC | 2012 |

TABLE 2
siRNA against Ebola Virus Protein 24

| VP24 | 25 mer | | 21mer |
|---|---|---|---|
| 1 | 5′-ggacgauacaaucuaauaucgccca-3′ | 9 | 5′-ggacgauacaaucuaauaudtdt-3′ |
| 2 | 5′-ggguugucuuaagcgaccucuguaa-3′ | 10 | 5′-ggguugucuuaagcgaccudtdt-3′ |
| 3 | 5′-guggaagguuuauugggcugguauu-3′ | 11 | 5′-guggaagguuuauugggcudtdt-3′ |
| 4 | 5′-gguuuauugggcugguauugaguuu-3′ | 12 | 5′-gguuuauugggcugguauudtdt-3′ |
| 5 | 5′-gcauggucaaugacaaggaaucucu-3′ | 13 | 5′-ccucgacacgaaugcaaagdtdt-3′ |
| 6 | 5′-ggauacaagaccagcugauugacca-3′ | 14 | 5′-ggauacaagaccagcugaudtdt-3′ |
| 7 | 5′-ggcugcuaacaaccaacacuaacca-3′ | 15 | 5′-gcugcuaacaaccaacacudtdt-3′ |
| 8 | 5′-caagaacccgacaaaucggcaauga-3′ | 16 | 5′-gaacccgacaaaucggcaadtdt-3′ |

TABLE 3
siRNA against Ebola Virus Protein 30

| VP30 | 25mer | | 21mer |
|---|---|---|---|
| 1 | 5′-gaauuaucgaggugaguaccgucaa-3′ | 9 | 5′-ucgaggugaguaccgucaadtdt-3′ |
| 2 | 5′-ccgucaaucaaggagcgccucacaa-3′ | 10 | 5′-cgucaaucaaggagcgccudtdt-3′ |
| 3 | 5′-ggagaguuuaacugauagggaauua-3′ | 11 | 5′-gagaguuuaacugauagggdtdt-3′ |
| 4 | 5′-caaggacucgcgcuuagcaaaucca-3′ | 12 | 5′-caaggacucgcgcuuagcadtdt-3′ |
| 5 | 5′-cuuguugacucugaucaagacggca-3′ | 13 | 5′-gacucugaucaagacggcadtdt-3′ |
| 6 | 5′-cucuaugugcugugaugacgaggaa-3′ | 14 | 5′-gugcugugaugacgaggaadtdt-3′ |
| 7 | 5′-ggcaagaucaggcagaaccuguucu-3′ | 15 | 5′-gcaagaucaggcagaaccudtdt-3′ |
| 8 | 5′-cucuaugugcugugaugacgaggaa-3′ | 16 | 5′-cuaugugcugugaugacgadtdt-3′ |

TABLE 4
siRNA against Ebola Virus Protein 35

| VP35 | 25mer | | 21mer |
|---|---|---|---|
| 1 | 5′-cugagcagcuaaugaccggaagaau-3′ | 9 | 5′-gagcagcuaaugaccggaadtdt-3′ |
| 2 | 5′-gcuaaugaccggaagaauuccugua-3′ | 10 | 5′-gcuaaugaccggaagaauudtdt-3′ |
| 3 | 5′-gcgacaucuucugugauauugagaa-3′ | 11 | 5′-gcgacaucuucugugauaudtdt-3′ |
| 4 | 5′-cuucauuggcuacuguugtgcaaca-3′ | 12 | 5′-cuucauuggcuacuguugudtdt-3′ |
| 5 | 5′-cgaauagcaaaccuugaggccagcu-3′ | 13 | 5′-gcaaaccuugaggccagcudtdt-3′ |
| 6 | 5′-ggguuugugcugagauggucgcaaa-3′ | 14 | 5′-gcaacucauuggacaucaudtdt-3′ |
| 7 | 5′-ggugaugacaaccggucgggcaaca-3′ | 15 | 5′-gaugacaaccggucgggcadtdt-3′ |
| 8 | 5′-caaccgcugcggcaacugaggcuua-3′ | 16 | 5′-caaccgcugcggcaacugadtdt-3′ |

TABLE 5
siRNA against Ebola Virus Protein 40

| VP40 | 25mer | | 21mer |
|---|---|---|---|
| 1 | 5′-ggguuauauuaccuacugcuccuca-3′ | 9 | 5′-gguuauauuaccuacugcudtdt-3′ |
| 2 | 5′-gguuauauugccuacugcuccuccu-3′ | 10 | 5′-gguuauauugccuacugcudtdt-3′ |
| 3 | 5′-cuccaucaaauccacucagaccaau-3′ | 11 | 5′-uccaucaaauccacucagadtdt-3′ |
| 4 | 5′-ccaugccagccacacaccaggcagu-3′ | 12 | 5′-ccaugccagccacacaccadtdt-3′ |
| 5 | 5′-cagcauucauccuugaagcuauggu-3′ | 13 | 5′-cauucauccuugaagcuaudtdt-3′ |
| 6 | 5′-ggcuuccucuaggugucgcugauca-3′ | 14 | 5′-gcuuccucuaggugucgcudtdt-3′ |
| 7 | 5′-cucaacaacggccgccaucaugcuu-3′ | 15 | 5′-caacggccgccaucaugcudtdt-3′ |
| 8 | 5′-ggcaaggcaaccaauccacuuguca-3′ | 16 | 5′-caaggcaaccaauccacuudtdt-3′ |

TABLE 6
siRNA against Ebola Virus L Protein

| LP | 25mer | | 21mer |
|---|---|---|---|
| 1 | 5′-ggaccaaugugaccuugucacuaga-3′ | 9 | 5′-gaccaaugugaccuugucadtdt-3′ |
| 2 | 5′-caacuacgcaacuguaaacucccga-3′ | 10 | 5′-cgcaacuguaaacucccgadtdt-3′ |
| 3 | 5′-ccaaguucuugagugauguaccagu-3′ | 11 | 5′-ccaaguucuugagugaugudtdt-3′ |
| 4 | 5′-cccaauucuucucaaggcacuguca-3′ | 12 | 5′-cccaauucuucucaaggcadtdt-3′ |
| 5 | 5′-cauuaaguacacaaugcaagaugcu-3′ | 13 | 5′-cauuaaguacacaaugcaadtdt-3′ |
| 6 | 5′-gugcucaagaagacuguguugauga-3′ | 14 | 5′-gugcucaagaagacugugudtdt-3′ |
| 7 | 5′-ggguagauuaaaucgaggaaacucu-3′ | 15 | 5′-ggguagauuaaaucgaggadtdt-3′ |
| 8 | 5′-ccaauuucaauguuaccacugaaca-3′ | 16 | 5′-caauuucaauguuaccacudtdt-3′ |

TABLE 7
siRNA Duplexes have been demonstrated potent to Anti-Ebola Virus Activity

| | ZEBOV | Sequence |
|---|---|---|
| | target | |
| EK-1 mod | L poly- | Sense 5′-GmUACGAAGCUmGUAUAmUAAAUU-3′, antisense 5′-UUUAmUAUACAGCUUCGmUACAA-3′ |
| | merase | |
| VP24-775 | VP24 | Sense 5′-GCUGAUUGACCAGUCUUUGAU-3′, antisense 5′-CAAAGACUGGUCAAUCAGCUG-3′ |
| VP24-978 | VP24 | Sense 5′-ACGGAUUGUUGAGCAGUAUUG-3′, antisense 5′-AUACUGCUCAACAAUCCGUUG-3′ |
| VP24-1160 | VP24 | Sense 5′-UCCUCGACACGAAUGCAAAGU-3′, antisense 5′-UUUGCAUUCGUGUCGAGGAUC-3′ |
| VP24-1160 | VP24 | Sense 5′-UCCmUCGACACGAAmUGCAAAGU-3′, antisense 5′-UUmUGCAUUCGUGUCmGAGmGAUC-3′ |
| mod | | |
| VP35-219 | VP35 | Sense 5′-GCGACAUCUUCUGUGAUAUUG-3′, antisense 5′-AUAUCACAGAAGAUGUCGCUU-3′ |
| VP35-349 | VP35 | Sense 5′-GGAGGUAGUACAAACAUUGdTdT-3′, antisense 5′-CAAUGUUUGUACUACCUCCdTdT-3′ |
| VP35-687 | VP35 | Sense 5′-GGGAGGCAUUCAACAAUCUAG-3′, antisense 5′-AGAUUGUUGAAUGCCUCCCUA-3′ |
| VP35-855 | VP35 | Sense 5′-GCAACUCAUUGGACAUAUUC-3′, antisense 5′-AUGAUGUCCAAUGAGUUGCUA-3′ |
| VP35-855 | VP35 | Sense 5′-GCAACmUCAUUGmGrArCrAmUAUUC-3′, antisense 5′-AUGAUmGUCCAAUGAmGUmUGCUA-3′ |
| mod | | |
| Luc | NA | Sense 5′-GAUUAUGUCCGGUUAUGUAAA-3′, antisense 5′-UACAUAACCGGACAUAAUCAU-3′ |
| Luc mod | NA | Sense 5′-GAmUmUAmUGmUCCGGmUmUAmUGmUAAA-3′, antisense 5′-UACAmUAACCGGACAmUAAmUCAU-3′ |

Sequences used in the non-human primates studies contain an m in front of the base that designates a 2′-O-methyl modification (unmodified versions do not have any 2′-O-methyl modification bases). mod = modification. Luc mod = modified luciferase. NA = not applicable.

TABLE 8
DNA Primers for RT-PCR Analysis of Viral RNA Levels

| | Primer Name | location | Primer Sequence | Base | Tm(° C.) | Size (bp) |
|---|---|---|---|---|---|---|
| VP35 | VP35 Forward | 3131-3152 | 5′-GAC AAC CAG AAC AAA GGG CAG G-3 | 22 | 64.2 | 222 |
| | VP35 Reverse | 3332-3353 | 5′-C GTT TGG GTT TGA CTG TTG CGC-3′ | 21 | 64.2 | |
| VP40 | VP40 Forward | 4481-2504 | 5′-GAG GCG GGT TAT ATT ACC TAC TGC-3′ | 24 | 65.2 | 304 |
| | VP40 Reverse | 4763-4785 | 5′-GA TCA GCG ACA CCT AGA GGA AGC-3′ | 23 | 66.6 | |
| VP30 | VP30 Forward | 8411-8434 | 5′-GAT CTG CGA ACC GGT AGA GTT TAG-3′ | 24 | 65.2 | 255 |
| | VP30 Reverse | 8644-8666 | 5′-CA GTA GGA ACG CGC ACT TGT GAG-3′ | 23 | 66.6 | |
| VP24 | VP24 Forward | 9983-10006 | 5′-CAG GGT AGT CCA ATT AGT GAC ACG-3′ | 24 | 65.2 | 243 |
| | VP24 Reverse | 10202-10226 | 5′-GTG AGG GCG CTC AAA GTG ATG TTC-3′ | 24 | 66.9 | |
| LP | L Forward | 11606-11628 | 5′-CG GAC GCT AGG TTA TCA TCA CC-3′ | 22 | 64.2 | 275 |
| | L reverse | 11839-11861 | 5′-GC TCA ACA GGA CAG AAC CCA TTG-3′ | 23 | 64.6 | |

1. A pharmaceutical composition, comprising at least 2 different siRNA molecules that target one or more conserved regions of an Ebola virus RNA or one or more conserved regions of different Ebola virus RNAs and a pharmaceutically acceptable carrier, wherein the Ebola virus is selected from the group consisting of Zaire ebolavirus (EBOV), Sudan ebolavirus (SUDV), Tai Forest ebolavirus (TAFV), Reston ebolavirus (RESTV), and Bundibugyo ebolavirus (BDBV), wherein the conserved regions of Ebola virus RNA are selected from the group consisting of VP24, VP30, VP35, VP40, and L Polymerase RNAs, wherein the pharmaceutically acceptable carrier comprises Histidine-Lysine co-polymer (HKP) or Spermine-Liposome-Cholesterol (SLiC), and wherein the siRNA molecules and the carrier form a nanoparticle.

2-3. (canceled)

4. The composition of claim 1, wherein the conserved regions comprise part of VP24, VP30, VP35, VP40, or L Polymerase RNA.

5-8. (canceled)

9. The composition of claim 1, wherein the siRNA molecules are selected from the siRNA molecules disclosed in Tables 2-6.

10. The composition of claim 39, wherein the siRNA molecules comprise VP24 (3): 5′-guggaagguuuauugggcugguauu-3′, VP35(4): 5′-cuucauuggcuacuguugtgcaaca-3′, and LP (5): 5′-cauuaaguacacaaugcaagaugcu-3′.

11. The composition of claim 39, wherein the siRNA molecules comprise VP24 (9): 5′-ggacgauacaaucuaauaudtdt-3′, VP35 (9): 5′-gagcagcuaaugaccggaadtdt-3′, and LP (9): 5′-gaccaaugugaccuugucadtdt-3′.

12. The composition of claim 1, wherein the siRNA molecules comprise VP24 (5): 5′-gcauggucaaugacaaggaaucucu-3′ and VP35 (5) 5′-cgaauagcaaaccuugaggccagcu-3′.

13. The composition of claim 1, wherein the siRNA molecules comprise VP24 (13): 5′-ccucgacacgaaugcaaagdtdt-3′ and VP35 (13): 5′-gcaaaccuugaggccagcudtdt-3′.

14. The composition of claim 1, wherein the siRNA molecules are selected from the group consisting of CT01, CT02, CT03, CT04, CT05, CT06, CT07, and CT08.

15. The composition of claim 1, wherein siRNA molecules are selected from the group consisting of PA01, PA02, PA03, PA04, PA05, PA06, PA07, PA08, PA09, and PA10.

16. A method of preventing or treating Ebola virus disease in a mammal, comprising administering a therapeutically effective amount of the composition of claim 1 to the mammal.

17-18. (canceled)

19. The method of claim 16, wherein the mammal is a human.

20. The method of claim 19, wherein the composition is administered intravenously.

21. The method of claim 20, wherein the therapeutically effective amount comprises about 1 mg of the siRNA molecules per kilogram of body weight of the human to about 5 mg of the siRNA molecules per kilogram of body weight of the human.

22-25. (canceled)

26. A method for testing the activity of the composition of claim 1, comprising testing the composition in a cell culture or an animal model.

27. The method of claim 26, wherein the cell culture comprises a Vero E6 cell culture.

28-29. (canceled)

30. The method of claim 26, wherein the animal model comprises an Ebola virus infected guinea pig model.

31. The method of claim 26, wherein the animal model comprises an Ebola virus infected non-human primate model.

32. (canceled)

33. The composition of claim 1, wherein the composition is lyophilized into a dry power.

34. A lipid nanoparticle (LNP) comprising a cationic lipid conjugated with cholesterol.

35. The LNP of claim 34, wherein the cationic lipid comprises a spermine head and an oleyl alcohol tail.

36. The LNP of claim 34, wherein the cationic lipid comprises a spermine head and two oleyl alcohol tails.

37. The composition of claim 1, wherein the siRNA molecules target VP30, VP40, or a part thereof.

38. The composition of claim 1, wherein the composition further comprises an Arg-Gly-Asp (RGD) peptide ligand.

39. A pharmaceutical composition, comprising at least 3 different siRNA molecules that target one or more conserved regions of an Ebola virus RNA or one or more conserved regions of different Ebola virus RNAs and a pharmaceutically acceptable carrier, wherein the Ebola virus is selected from the group consisting of Zaire ebolavirus (EBOV), Sudan ebolavirus (SUDV), Tai Forest ebolavirus (TAFV), Reston ebolavirus (RESTV), and Bundibugyo ebolavirus (BDBV), wherein the conserved regions of Ebola virus RNA are selected from the group consisting of VP24, VP30, VP35, VP40, and L Polymerase RNAs, wherein the pharmaceutically acceptable carrier comprises Histidine-Lysine co-polymer (HKP) or Spermine-Liposome-Cholesterol (SLiC), and wherein the siRNA molecules and the carrier form a nanoparticle.

40. A method of preventing or treating Ebola virus disease in a mammal, comprising administering a therapeutically effective amount of the composition of claim 39 to the mammal.
